(19) United States
(12) Patent Application Publication
(10) Pub. No.: US 2005/0133025 A1
(43) Pub. Date: Jun. 23, 2005

(54) Inhalator and method of manufacturing same

(76) Inventors: Juha Laiho, Kangasala (FI); Vesa Junkkarinen, Kontiolahti (FI); Veikko Kosunen, Joensuu (FI)

Correspondence Address: YOUNG & THOMPSON, 745 SOUTH 23RD STREET, 2ND FLOOR, ARLINGTON, VA, 22202, US

(21) Appl. No.: 10/986,842

(22) Filed: Nov. 15, 2004

Related U.S. Application Data
(63) Continuation of application No. PCT/FI03/00367, filed on May 13, 2003.

(30) Foreign Application Priority Data
May 14, 2002 (FI) 20020909

(51) Int. Cl. C23C 16/00; A61M 11/00; C25D 5/00

(52) U.S. Cl. 128/200.23; 427/248.1; 205/80

(57) ABSTRACT

An inhalator, an inhalator component and a method for manufacturing an inhalator component. The inhalator and inhalator component have at least one surface made of polymer material that includes a coating layer. The coating layer substantially reduces moisture penetration through the surface and lowers the specific electric resistance of the surface.

### BACKGROUND OF THE INVENTION

The invention relates to an inhalator having at least one surface made of polymer material.

The invention further relates to an inhalator component that is at least partly made of polymer material.

The invention further relates to a method for manufacturing an inhalator component.

An inhalator administers a pharmaceutical agent to inhaled air. The user of the inhalator breathes in air through the inhalator and at the same time a specific amount of the pharmaceutical agent is mixed with the airflow passing through the inhalator.

The pharmaceutical agent in an inhalator is often in powder form, in which case the inhalator is a powder inhalator, but inhalators are also known, in which the pharmaceutical agent is dissolved in liquefied carrier gas.

The pharmaceutical agent is usually arranged in a drug container in the inhalator. In some inhalators, the drug container contains a predosed dosage of the pharmaceutical agent that passes with the inhaled air to the organ system of the inhalator user; in such a case, the inhalator has several separate drug containers containing the pharmaceutical agent that are arranged in a magazine-like manner in several separate, small drug containers. In some other inhalators, a specific amount of the pharmaceutical agent is administered with dosing means from the drug container to a specific chamber in the inhalator, from which the pharmaceutical agent is mixed with inhaled air.

Which ever of the above principles the operation of the inhalator is based on, the inhalator comprises means for removing the pharmaceutical agent from the drug container and for arranging the pharmaceutical agent to mix with the airflow passing through the inhalator.

The inhalator can be a disposable one, in which case it becomes litter after the pharmaceutical agent in it has run out, or it can be refilled with the pharmaceutical agent after it has run out.

The manufacturing materials of the inhalator components must meet specific requirements provided by the authorities, such as FDA. A considerable number of the components in a modern inhalator are made of polymer materials, because their shaping properties, lightness and price are superior to other materials. Polymer materials do, however, have some problems. The handling of a pharmaceutical agent in powder form in particular is demanding. Moisture entering the inhalator or drug container causes the powdery agent to agglomerate and arch, which in turn reduces the ability of the pharmaceutical agent to mix with the air flowing through the inhalator. Moisture penetrates through polymer materials due to their internal structure.

In inhalators containing a pharmaceutical agent dissolved in liquefied carrier gas, moisture may essentially change the pharmaceutical agent concentration of the solution or cause other corresponding phenomena that alter the amount of pharmaceutical agent administered by the inhalator.

In addition, electrostatic charges are easily generated between a powdery pharmaceutical agent and the inhalator or drug container surfaces, which cause the pharmaceutical agent to accumulate on the surfaces. In other words, the pharmaceutical agent intended to mix with air does not entirely mix with the airflow, but part of it remains on the surfaces. On the other hand, the agent sticking to the surfaces due to the charging may detach in an uncontrolled manner and cause an overdose.

Due to the above-mentioned reasons, the deviation of the inhaled dose of the pharmaceutical agent increases. On the other hand, the dose of the pharmaceutical agent released from the inhalator may change due to moisture in the air, for instance, whereby the deviation of the dose from the inhalator varies. With time, the inhalator may also block or must be discarded, because the dose of the pharmaceutical agent provided by it is outside the therapeutic range. To avoid situations like this, the storage and usage time of the inhalator needs to be limited. The inhalator may need to be discarded before the pharmaceutical agent is entirely used. In addition, the variance and slow decrease in inhalator power is very uncomfortable for the user.

In this respect, the properties of plastics that are competitive in price and processing costs and accepted for said use are poor, because they are permeable to moisture and their specific electric resistance is high.

To prevent moisture from getting to the pharmaceutical agent, drying cartridges, such as silica gel packs, are introduced to the inhalator to absorb the moisture inside the protective casing of the inhalator or diffusing through openings or wall structure to it. The generation of static electricity is reduced by mixing to the polymer material of the inhalator components a filling agent, such as metal particles or carbon black, that reduces its specific electric resistance. Various production engineering methods are also known that are used to try to provide a product without an electric charge. However, an entirely satisfactory solution has not been found for the above-mentioned problems.

### BRIEF DESCRIPTION OF THE INVENTION

It is an object of the present invention to provide a novel and improved inhalator, inhalator component and method for manufacturing one.

The inhalator of the invention is characterized in that said surface comprises a coating layer that substantially reduces moisture penetration through said surface and lowers the specific electric resistance of said surface.

The inhalator component of the invention is characterized in that at least some of the component surfaces made of polymer material are coated with a coating layer that substantially changes moisture penetration through said surface and lowers the specific electric resistance of said surface.

Further, the method of the invention is characterized by producing on at least one surface of the component a coating layer that substantially reduces moisture penetration through said surface and the specific electric resistance of said surface.

Further, the idea of a preferred embodiment of the invention is that the coating is at least mainly made of metal or alloy. Further, the idea of a second preferred embodiment of the invention is that the coating is at least mainly made of amorphous carbon. Further, the idea of a third preferred embodiment of the invention is that the coating is at least mainly made of a ceramic material. Further, the idea of a fourth preferred embodiment of the invention is that the coating is made of polymer or polymer composite. Further, the idea of a fifth preferred embodiment of the invention is that the coating is utilized as an electric conductor that is arranged to conduct electric energy to the electric components of the inhalator. The invention provides the advantage that the moisture penetration rate of the inhalator component and the generation of static electric on its surface is reduced, whereby the dosage of the pharmaceutical agent administered by the inhalator varies less than in the prior-art inhalators. In addition, the usage time of the inhalator or its pharmaceutical agent is lengthened, thus also lengthening the usage time of a disposable inhalator. The pharmaceutical industry can utilize ever-increasing lot sizes and lengthening storage time for instance in that the pharmaceutical agent can be prepared in larger batches. On the other hand, the user can store products longer without their effect becoming weaker. CVD and PVD methods produce thin coatings from almost any initial material, and they can be used to provide coatings that are exactly tailored for their purpose both in material and in the thickness of the coating layer. The sol-gel method only needs simple equipment and its costs are very low. Electroplating is a fast and simple method. A coating layer made at least mainly of metal or alloy reduces both moisture penetration and electrostatic charges. A coating layer made at least mainly of amorphous carbon or ceramic material is inert, mechanically and chemically stable, biocompatible and very homogeneous. Mechanical and chemical stabilities are significant properties in dosing devices from which no particles should detach to the administered pharmaceutical agent. In addition, the friction coefficient of coating layers made at least mainly of amorphous carbon or ceramic material is typically low, which property may be advantageous in the surfaces of the inhalator that move against each other. The properties of a coating made of polymer material can be tailored as necessary to reduce moisture penetration and electrostatic charges. Utilizing the coating layer as an integrated conductor reduces firstly the space required by the electric conductors in the inhalator, secondly the assembly work caused by the handling of the conductors, and thirdly the number of surfaces and shapes caused by the separate electric conductors problematic in view of hygiene. Arranging the coating layer suitably inside the protective casing of the inhalator, for instance, protects the electric components in the inhalator from the electromagnetic disturbances coming from outside the inhalator, in other words, the coating layer forms at least part of the EMC shielding of the inhalator.

### BRIEF DESCRIPTION OF THE FIGURES

The invention will now be described in greater detail by means of preferred embodiments and with reference to the attached drawings, in which

FIG. 1 is a partly cross-sectional schematic side view of an inhalator of the invention,

FIG. 2 is a partly cross-sectional schematic side view of a second inhalator of the invention,

FIG. 3 is a schematic view of a drug container of the invention, and

FIG. 4 is a partly cross-sectional schematic side view of equipment implementing the method of the invention.

### DETAILED DESCRIPTION OF THE INVENTION

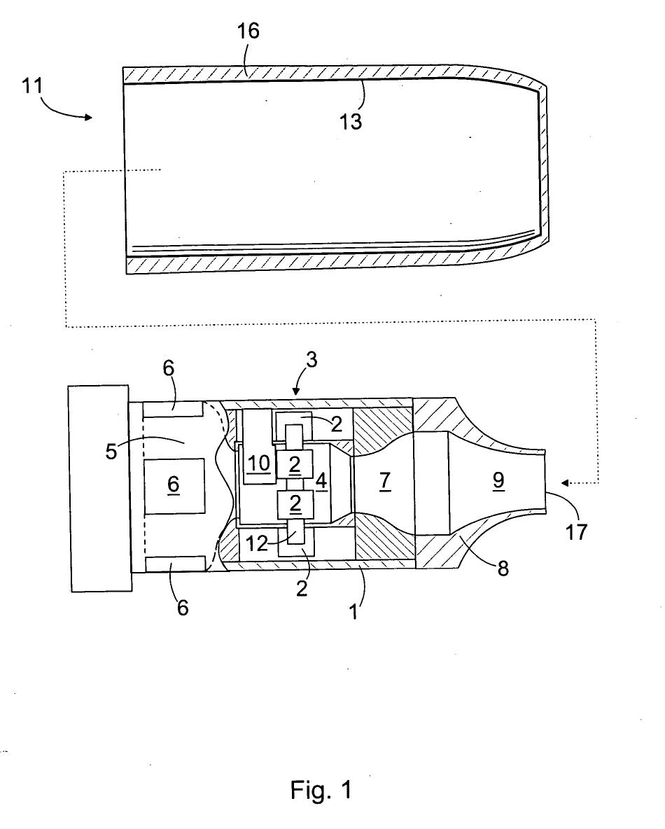

FIG. 1 is a partly cross-sectional schematic side view of an inhalator of the invention. The powder inhalator shown in FIG. 1 comprises a body 1 that is preferably made of polymer material by moulding or injection moulding or another corresponding method. The drug containers 2 of the inhalator are arranged inside the body 1. The shown inhalator comprises several drug containers arranged in a magazine 3. The magazine 3 also comprises a circular magazine body 12 that connects the drug containers 2 to each other. The body 12 is made of polymer material.

Each drug container 2 comprises a closed space, to which a pre-dosed amount of a pharmaceutical agent is arranged. The amount of the pharmaceutical agent is pre-dosed either in such a manner that the contents of one drug container produces the therapeutic effect in the user or in such a manner that the amount of the pharmaceutical agent administered by the inhalator in one inhalation can be adjusted. This way, small users, such as children, whose therapeutic range is reached with a smaller dosage of the pharmaceutical agent, can inhale a smaller dose than adults or large users, whose therapeutic range is reached with a larger dose. For this, the inhalator has means, such as an adjusting wheel or the like, with which the user defines the number of drug containers to be used/emptied in one inhalation.

A mixing space 4, in which the pharmaceutical agent mixes with air, is located inside the drug container magazine 3. Means 10 for emptying the drug containers are arranged to the mixing space 4. Said means 10 open the closed space of the drug container 2 and transfer the pharmaceutical agent in the drug container 2 to the mixing space. The means 10 are known per se and numerous different variations exist of them, so they are not discussed herein in detail. The user of the inhalator controls the means 10 by using control means that are not shown in the figure to simplify the presentation.

In a second inhalator of the invention, the pharmaceutical agent is arranged in only one drug container. Only a specific amount of the pharmaceutical agent is administered from the drug container for mixing with air. In other words, the drug container is filled with more than one dose of the pharmaceutical agent.

An air inlet 5, shown mainly by a dashed line in the figure, leads to the mixing space 4. In the presented embodiment, the air inlet openings 6 of the air inlet 5 are arranged at regular intervals around the body, but they can naturally be placed in some other manner on the body 1 of the inhalator. The pharmaceutical agent is mixed with air coming through the air inlet 5 to the mixing space 4. The inhalator further comprises an outlet 7 that is also connected to the mixing space 4. The pharmaceutical agent that is mixed with air in the mixing space 4 flows out of the mixing space 4 through the outlet 7. The inhalator also has a detachable mouthpiece 8 with a flow channel 9 connected to the outlet 7. The air containing the pharmaceutical agent flows to the pulmonary organs of the user through the mouthpiece 8. During inhalation, the mouthpiece is inserted into the mouth of the user of the inhalator. In another embodiment, the mouthpiece 8 is inserted to the nose of the user.

The air inlet openings 6, air inlet 5, mixing space 4, outlet 7 and the flow channel 9 of the mouthpiece form an air channel through the inhalator. During inhalation, at least part of the air inhaled by the user of the inhalator flows through the air channel.

The inhalator also comprises a detachably attachable protective casing 11, which in FIG. 1 is shown detached from the inhalator. The protective casing 11 is arranged on the body 1 of the inhalator when the inhalator is not used, and is removed from the body 1 immediately before inhalation. When the protective casing 11 is arranged on the body 1, it is essential that it covers the air inlet openings 6 and the opening 17 of the mouthpiece flow channel. The protective casing 11 can be fastened on the body 1 with a compression, snap or screw joint or another corresponding fastening method that is simple and reliable.

The protective casing 11 comprises a body 16 that is made of polymer material preferably by moulding, injection moulding, compression, thermoforming or another corresponding method. The inner surface of the body 16 of the protective casing is coated with a coating layer 13 that reduces moisture penetration through the wall of the protective casing. In addition to this, the coating layer 13 protects the electric components inside the inhalator from electromagnetic disturbances coming from outside the inhalator, i.e. the coating layer 13 is part of the EMC shielding. It should be noted that said electric components are not shown in FIG. 1, but they are typically related to locking devices, for instance, by which misuse of the inhalator can be prevented, or to alarms that remind the user to take the dose on time, or to corresponding devices.

The body 16 has been the substrate being coated in the coating process. The material of the coating layer 13 is for instance metal, such as stainless steel, amorphous carbon or ceramic polymer mixture. The coating layer 13 can alternatively be arranged on the outer surface of the protective casing 11. For instance, a metal coating layer on the outer surface of the protective casing 11 creates an aesthetically pleasant and high-quality impression on the inhalator.

The coating layer 13 can be made with one of the following coating methods: CVD (chemical vapour deposition) method, PVD (physical vapour deposition) method, sol-gel method, electroplating, ALD (atomic layer deposition) method or modifications based thereon. In the methods, it is possible to use so low process temperatures that the coating of plastic parts is possible. With said methods, it is possible to form coating layers that are suitable in thickness, such as thin coatings in the range of micrometers. In addition, the methods are suitable for use with numerous coating materials and coated substrate materials and for coating substrates that are complex in shape. In the following, the main features of each method are described. It should be noted that the methods are known per se, but have been applied mainly to the manufacture of hard, wear-resistant coatings or optically advantageous coatings.

CVD (Chemical Vapour Deposition)

The CVD method with its various modifications is especially suited for making DLC (diamond-like carbon) coatings, i.e. diamond-like coatings of amorphous carbon, i.e. amorphous diamond coatings, on a substrate of polymer material, for instance. It should be noted in this context that in this application, the term polymer material refers to materials made of plastics, plastic mixtures and plastic composites.

The DLC coating comprises amorphous carbon having similar linkages as a diamond. The DLC coating is known per se and has been applied, among other things, as a wear- and corrosion-reducing coating and a friction-reducing coating.

The manufacture of the DLC coating is based on a method generally known as PCVD (plasma chemical vapour deposition) or PACVD (plasma-assisted chemical vapour deposition) or PECVD (plasma-enhanced chemical vapour deposition). In this, the component being coated, in this case the protective casing 11, is placed on an electrode capacitively coupled to a high-frequency source. The electrode is, in turn, in a vacuum chamber. The parts of the substrate that need not be coated are covered. A plasma field is generated using microwaves or an electrical field in the chamber. The energy that initiates the actual coating, i.e. the fastening of carbon to the surface of the substrate, is generated when plasma ions and electrons impact. A momentary and very local high temperature and pressure cause the carbon atoms to link as in a diamond.

Various gases or gas mixtures can be fed in to the vacuum chamber to adjust the properties of the coating. The coating temperature is in the range of 100° C. The thickness of the coating layer 13 is typically 1 to 4 μm. The process can be manual, automatic or a combination thereof. The shape of the surface to be coated is preferably taken into account in the design of the vacuum chamber and electrodes to achieve optimum coating conditions and an optimum coating layer 13.

One advantage of the plasma-assisted CVD method is that even very complex surfaces can be coated with it as well as heat-sensitive polymer materials.

PVD (Physical Vapour Deposition

PVD methods are processes based on vaporised coating material, in which at least one non-gaseous initial material is first vaporised and then the atoms, molecules or ions of the vaporised initial material are allowed to form a solid coating layer on the surface of the substrate. The vaporisation of the initial material can be produced for instance by thermal vaporisation, sputtering, electric arc vaporisation or chemical vapour or gases. High frequency sputtering is used when the substrate is a substantially electrically non-conductive material, such as the protective casing 11 made of polymer material in the present case.

The PVD method comprises three main phases: 1) vaporisation of the coating material, 2) transfer of the coating material to the substrate being coated, and 3) deposition of the coating material and growth of the coating on the substrate. The deposition can contain a reactive phase, in which the vaporised coating material reacts with at least one other vaporised coating material and forms a chemical compound, such as nitride, oxide, carbide or carbon nitride. In practice, the coating material can be any known inorganic coating material; it is also possible to use it with a few organic coating materials. In most cases, the coating material is metal, ceramics, metal nitride or the like. The PVD method can also be applied to making diamond-like coatings. In addition, it is possible to use a few polymers, such as Teflon PTFE, or other special plastics, which endure plasma bombardment, as the coating material in the method. The thickness of the coating layer 13 is typically 1 to 2 μm. Electrically conducting material, such as electrically conducting particles or fibres, can be mixed with a non-conducting coating material per se to produce a sufficient electrical conductivity.

ALD (Atomic Layer Deposition)

The ALD method is a vacuum deposition method known per se, in which the coating layer is formed one atomic layer at a time. An advantage of the method is that the properties of the coating layer can be adjusted to exactly correspond to the property profile set for the coating layer. The method is known as the manufacturing technology of certain display devices.

Sol-Gel Method

In the sol-gel method, a thin, solid coating layer is formed on the surface of the substrate from a liquefied raw material. Known solutions of the method include hydrophobic coatings as coatings for optical components, for instance, anti-corrosion coatings and wear-reducing coatings. The sol-gel method is based on hydrolysis and condensation reactions of organometallic compounds in alcohol solutions. Inorganic or metal-organic agents, such as metal alkoxides, are used as the initial material. Other suitable initial materials include metal carboxylates, metal alcylamides, amorphous and crystalline colloid sol solutions and organic or inorganic hybrids. The sol-gel method produces a ceramic polymer coating.

The spreading processes of the sol-gel coating can be divided into four main categories: 1) spin processes, 2) dip processes, 3) roll coating processes, and 4) injection processes.

In a spin process, the coating liquid is administered on the substrate to be coated, after which the substrate is made to spin, whereby the liquid spreads by means of the centrifugal force on the surface to be coated. After this, the coating layer is thinned by vaporising the solvent in it.

In a dip process, the substrate to be coated is dipped in the coating liquid and raised from it at a specific speed at specific temperature and atmospheric conditions, after which the solvent is vaporised from the coating liquid remaining on the surface of the substrate and a solid coating layer remains. The final hardening of the sol-gel coating is done using external energy. The energy is usually directed to the coating by heat treatment in an oven, or as IR or UV radiation.

In a roll coating process, the coating liquid is spread on the surface of the substrate with one or more rolls. After the solvent is vaporised, a solid coating layer remains. The sol-gel coating can also be applied with a tampo printing principle.

The coating layer 13 of the protective casing 11 of the inhalator in FIG. 1 can be formed with the spin process, for instance.

The coating layer 13 can also be made by electroplating assuming that the surface to be coated is made of an electrically conducting material. In electroplating, the part to be coated is immersed in a metalline aqueous solution. The part to be coated acts as cathode and the metal to be precipitated, or in some cases, an insoluble anode, acts as the anode. The part to be coated or at least the surface to be coated can be polymer material containing butadiene.

The coating layer 13 can be made to a point of the inhalator, where two material surfaces move, for instance slide or roll, relative to each other. The coating layer 13 can then alter the friction coefficient between the surfaces. A coating layer 13 comprising PTFE or amorphous carbon or ceramic material, for instance, can lower said friction coefficient, in which case the operation of the inhalator requires less power. It is then easier than before for the elderly or persons with less strength to use the inhalator.

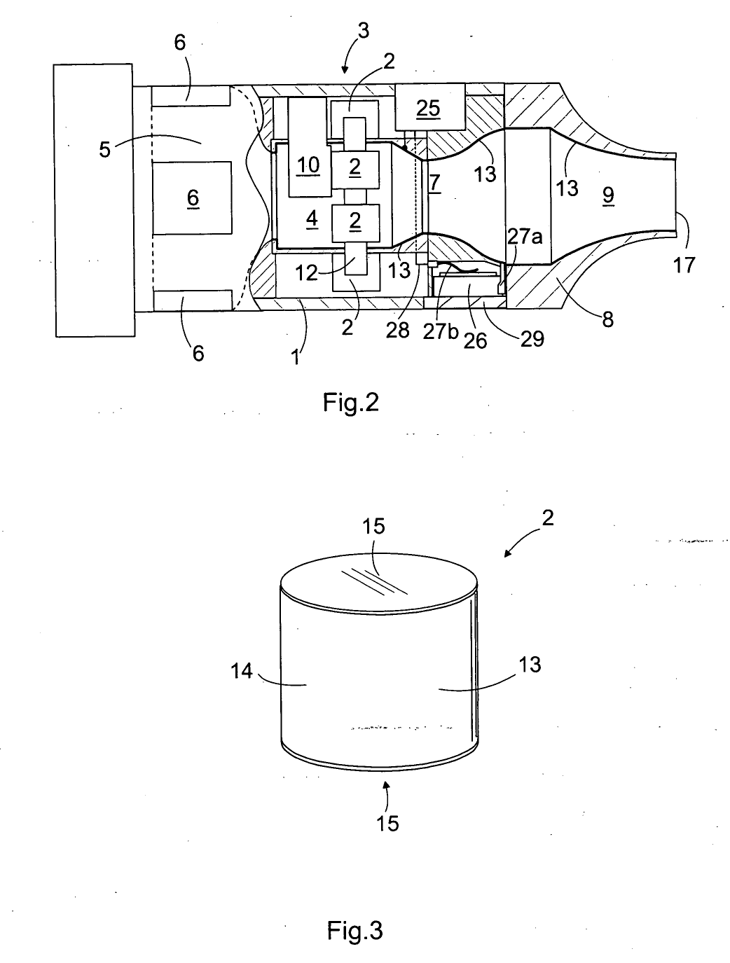

FIG. 2 is a partly cross-sectional schematic side view of a second inhalator of the invention. The inhalator is mainly similar in structure and operation to the inhalator shown in FIG. 1, but the coating layer 13 is now arranged on the inner surfaces of the mixing space 4, outlet 7 and flow channel 9 of the mouthpiece, i.e. on the surfaces that during inhalation are in contact with the pharmaceutical agent.

The coating layer 13 is now made of metal that increases the ability of the coated surface to discharge electric surface charges, i.e. it lowers the specific electric resistance of the surfaces, in other words, is an antistatic agent. Surfaces coated with an antistatic agent discharge electric charges efficiently. In addition, the coating surface 13 reduces moisture penetration in the coated surface and the penetration of gases, such as oxygen. The sticking and accumulation of the powdery pharmaceutical agent on the surfaces is reduced. This way, only a small amount of the pharmaceutical agent remains in the inhalator and correspondingly, the amount of the pharmaceutical agent exiting with air from the inhalator increases.

The specific resistance of the coating layer 13 can be so low that it can be utilised as an electric conductor that is arranged to conduct electric energy between the electric components arranged in the inhalator. The inhalator shown in FIG. 2 comprises an electrical alarm 25. The alarm 25 gives an alarm to the user of the inhalator every time the pharmaceutical agent should according to treatment instructions be taken. The alarm receives the electrical energy it needs to operate from a battery 26 that is placed in a battery compartment closable by a cover 29. The poles of the battery 26 are connected to contact elements 27a, 27b. The first contact element 27a is connected through the coating layer 13 to the alarm 25. Correspondingly, the second contact element 27b is connected by a current conductor 28 to the alarm 25.

The coating layer 13 can naturally be utilised as part of the electric circuit of several electric components, such as the earth potential of the inhalator.

A conductor integrated to the coating layer 13 reduces the space required by the separate electric conductors in the inhalator. At the same time, it is possible to reduce the hygiene problems caused by the separate conductors.

The coating layer 13 is made as already described in connection with FIG. 1.

FIG. 3 is a schematic view of a drug container of the invention. The drug container 2 is detachably attached to the inhalator: when the pharmaceutical agent runs out, the container can be detached from the inhalator and replaced with a new drug container 2.

The drug container 2 shown in FIG. 3 typically comprises a cylinder part 14 made of plastic and closed at both ends with a film 15 that is typically a metal-coated material or metal. The outer surface of the cylindrical part 14 is coated with the coating layer 13 that substantially reduces the penetration rate of moisture through the cylindrical part 14 to the drug container 2.

The coating layer 13 can be made with any of the coating methods described above. The material of the coating layer 13 is metal, ceramics, or amorphous carbon, for instance.

Alternatively or in addition to the coating of the outer surface, it is naturally also possible to coat the inner surface of the cylindrical part 14. The inner surface can be coated in such a manner that it either reduces the moisture penetration rate or the specific electric resistance or preferably both.

The film 15 can also be made of polymer material that is coated with the CVD, PVD or sol-gel method or by electroplating.

The drug container 2 shown in FIG. 3 is only a general example: it is apparent that the drug container can also be shaped and constructed in another manner. The drug container 2 can be entirely made of the same polymer material that is coated with a solid coating layer 13. The drug container 2 can comprise functional parts, such as opening and closing channels, through which a pharmaceutical agent can be added to the container or through which a pharmaceutical agent is administered from the drug container to the mixing space.

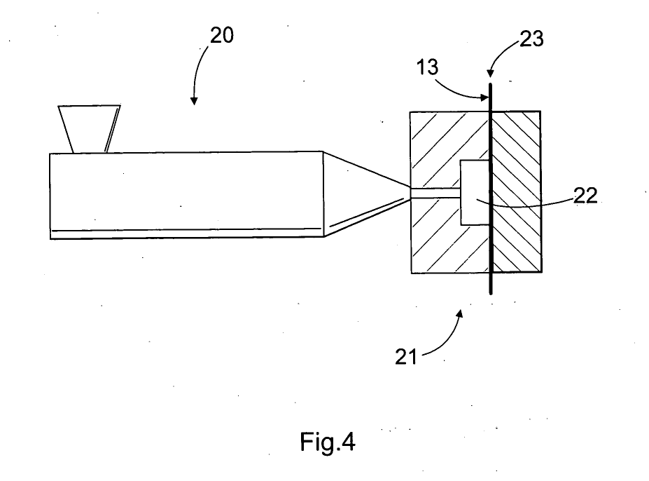

FIG. 4 is a partly cross-sectional schematic side view of equipment implementing the method of the invention. The coating layer 13 can be made based on the sol-gel method as follows: the inhalator component to be coated is made with the IMD (in mould decorating) technique. This is an application of injection-moulding, in which the component is shaped by injecting the raw material with injection-moulding means 20 to a mould cavity 22 of an injection mould 21, to which an IMD film 23 is arranged that typically comprises a carrier film and attached to it, a coating layer 13 or its precursor on top of each other. In the present embodiment, the coating layer 13 is made with the sol-gel method. When polymer material is injected to the mould cavity 22, the coating layer 13 of the IMD film 23 attaches to the surface of the component. After this, the mould 21 is opened and the component and the coating layer 13 attached to it are removed for a new work cycle. At the same time, the carrier film of the IMD film is removed from the mould cavity 22 and a new IMD film 23 comprising a coating layer 13 is set in. The IMD film 23 can be wound to a roll, from which it is unwound in necessary length between the mould halves. The method is fast and well applicable to large-scale production.

The coating layer 13 of the IMD film can naturally be made in other ways, too, for instance with the CVD and PVD methods described in this application or by electroplating. The coating layer 13 can have a crosslinking material component that is cross-linked after injection moulding by using external energy. Such a coating layer 13 can still be elastic at the injection-moulding stage and hard after cross-linking.

It is obvious to a person skilled in the art that while the technology advances, the basic idea of the invention can be implemented in many different ways. The invention and its embodiments are thus not restricted to the examples described above, but can vary within the scope of the claims. Thus, the coating layer 13 can be arranged on other surfaces than those mentioned above, for instance on the outer surface of the inhalator body 1. When the aim is to reduce moisture penetration in particular, a diamond-like deposition, stainless steel, gold or metal oxides can be used. Surface charges can be reduced by a doped diamond-like deposition, stainless steel, gold coating or metal oxides made conductive by doping. The thickness of the coating layer 13 is preferably 1 to 5 μm.

1. An inhalator having at least one surface made of polymer material, wherein said surface comprises a coating layer that substantially reduces moisture penetration through said surface and lowers the specific electric resistance of said surface.

2. An inhalator as claimed in claim 1, wherein the coating layer is arranged to a drug container.

3. An inhalator as claimed in claim 1, wherein the coating layer is arranged to a protective casing.

4. An inhalator as claimed in claim 1, wherein the coating layer is made of amorphous carbon.

5. An inhalator as claimed in claim 1, wherein the coating layer comprises metal or alloy.

6. An inhalator as claimed in claim 1, wherein the coating layer comprises a ceramic material.

7. An inhalator as claimed in claim 1, wherein the coating layer comprises polymer material.

8. An inhalator as claimed in claim 1, wherein the coating layer is at most 5 μm.

9. An inhalator as claimed in claim 1, wherein the inhalator is a disposable inhalator.

10. An inhalator as claimed in claim 1, wherein the coating layer is arranged to a surface that moves in relation to another surface of the inhalator and that the coating layer alters the friction coefficient between said surfaces.

11. An inhalator as claimed in claim 1, wherein the coating layer is arranged to be an electric conductor that is coupled with the electric components of the inhalator.

12. An inhalator as claimed in claim 1, wherein the coating is arranged to operate as an EMC shield element.

13. An inhalator component that is at least partly made of polymer material, wherein at least some of the surfaces of the component made of polymer material are coated with a coating layer that substantially reduces moisture penetration through said surface and lowers the specific electric resistance of said surface.

14. An inhalator component as claimed in claim 13, wherein the component is a drug container.

15. An inhalator component as claimed in claim 14, wherein the drug container is detachably attached to the inhalator.

16. An inhalator component as claimed in claim 14, wherein the drug container is refillable with a pharmaceutical agent.

17. An inhalator component as claimed in claim 13, wherein the component is an inhalator channel, through which the inhaled air flows.

18. An inhalator component as claimed in claim 13, wherein the component is a body of the inhalator.

19. An inhalator component as claimed in claim 13, wherein the component is a protective casing of the inhalator.

20. An inhalator component as claimed in claim 13, wherein the component is a dosage element of the pharmaceutical agent.

21. A method for manufacturing an inhalator component by producing on at least one surface of the component a coating layer that substantially lowers moisture penetration through said surface and the specific electric resistance of the surface.

22. A method as claimed in claim 21, wherein the coating layer comprises amorphous carbon.

23. A method as claimed in claim 21, wherein the coating layer comprises metal or alloy.

24. A method as claimed in claim 21, comprising steps of manufacturing an IMD film that comprises a coating layer having a lower moisture penetration and/or specific electric resistance than said polymer material, arranging said IMD film in an injection mould cavity providing the shape of the component, injecting polymer material into the mould cavity in such a manner that the IMD file attaches to the polymer material, and allowing the polymer material to harden, after which the component and the attached coating layer can be removed from the mould cavity.

25. A method as claimed in claim 24, wherein said coating layer is made with a CVD method.

26. A method as claimed in claim 24, wherein said coating layer is made with a PVD method.

27. A method as claimed in claim 24, wherein said coating layer is made with a sol-gel method.

28. A method as claimed in claim 24, wherein said coating layer is made by electroplating.

29. A method as claimed in claim 24, wherein said coating layer is made with an ALD method.
